(19) United States
(12) Patent Application Publication
(10) Pub. No.: US 2021/0133454 A1
(43) Pub. Date: May 6, 2021

(54) TERMINAL DEVICE, INFORMATION PROCESSING METHOD, AND NON-TRANSITORY STORAGE MEDIUM RECORDING PROGRAM

(71) Applicant: OMRON HEALTHCARE Co., Ltd., Kyoto (JP)

(72) Inventors: Yuki SAKAMOTO, Kyoto (JP); Yoshihito NAKANISHI, Kyoto (JP); Takanobu YAMAUCHI, Kyoto (JP)

(21) Appl. No.: 17/148,899

(22) Filed: Jan. 14, 2021

Related U.S. Application Data
(63) Continuation of application No. PCT/JP2019/026086, filed on Jul. 1, 2019.

(30) Foreign Application Priority Data
Jul. 20, 2018 (JP) 2018-136992

(51) Int. Cl. G06K 9/00; G06F 3/14; H04N 5/232

(52) U.S. Cl. CPC G06K 9/00671; G06F 3/14; G16H 30/20; G06K 2209/03; H04N 5/232945

(57) ABSTRACT

A terminal device according to an aspect includes an image capture device configured to capture an image of an electronic device to generate a captured image, the electronic device being in a state where display content including a display element is displayed on a display screen, a recognition unit configured to recognize, from the captured image, the display element in the display content, a model information acquisition unit configured to acquire model information indicating a model of the electronic device, a descriptive information acquisition unit configured to acquire descriptive information including descriptive text of the display element and associated with the model information, and a presentation unit configured to present the descriptive text in the descriptive information.

### CROSS-REFERENCE TO RELATED APPLICATIONS

This application is the U.S. national stage application filed pursuant to 35 U.S.C. 365(c) and 120 as a continuation of International Patent Application No. PCT/JP2019/026086, filed Jul. 1, 2019, which application claims priority from Japanese Patent Application No. 2018-136992, filed Jul. 20, 2018, which applications are incorporated herein by reference in their entireties.

### TECHNICAL FIELD

The present invention relates to technology that provides, for example, description related to an electronic device to a user.

### BACKGROUND ART

In a case where there is something unknown about an electronic device, such as a case where an unknown display element (a pictogram, for example) is displayed on a display screen of the electronic device, a user generally accesses a website associated with the electronic device by using a terminal device such as a smart phone, and refers to an electronic manual (may also be referred to as an operation guide) of the electronic device.

In the method described above, there are many procedures until a user reaches desired information (for example, descriptive text describing meaning of a pictogram). For example, to access the website, an operation such as a search is necessary. Further, it is necessary to search for a location of the desired information in the electronic manual. Although a keyword search is available in the electronic manual, it is still necessary to input a keyword to perform a search. Additionally, as for a display element such as a pictogram, a user does not know which keyword is to be used to perform a search, and even when the keyword search is available in the electronic manual, a user searches several pages for the desired information. Further, to perform a search for reaching a corresponding electronic manual, information such as a correct product name or model may be necessary, and often such information cannot be obtained easily.

For example, Patent Document 1 discloses a terminal device that displays an electronic manual. This terminal device displays an image of a vehicle including a plurality of points, and superimposes a speech bubble indicating an item of a site corresponding to a point tapped by a user on the image to display the speech bubble. Then, in response to the user tapping the speech bubble, the terminal device displays manual information according to the item described above.

### CITATION LIST

### Patent Literature

Patent Document 1: JP 2016-162155 A

### SUMMARY OF INVENTION

### Technical Problem

In Patent Document 1, it is easy to access manual information. However, it is necessary to search manual information for desired information, and a user cannot easily reach the desired information.

The present invention has been made focusing on the above-described circumstances, and an object of the present invention is to provide a terminal device, an information processing method, and a non-transitory storage medium recording a program that enable a user to easily obtain description of a display element displayed in an electronic device.

### Solution to Problem

The present invention adopts the following configurations to solve the above-described problem.

A terminal device according to an aspect includes an image capture device configured to capture an image of an electronic device to generate a captured image, the electronic device being in a state where display content including a display element is displayed on a display screen, a recognition unit configured to recognize, from the captured image, the display element in the display content, a model information acquisition unit configured to acquire model information indicating a model of the electronic device, a descriptive information acquisition unit configured to acquire descriptive information including descriptive text of the display element and associated with the model information, and a presentation unit configured to present the descriptive text in the descriptive information.

According to the above-described configuration, when a user captures an image of an electronic device by using the image capture device of the terminal device, a display element in display content displayed in the electronic device is recognized, and descriptive text of the recognized display element is presented to the user. In this way, the user performs a simple operation including the image capture of the electronic device to obtain the description of the display element. Thus, the user can easily obtain the description of the display element displayed in the electronic device.

According to an aspect, the display element may be a pictogram. According to this configuration, even when the display element is a pictogram that is difficult to express by using a keyword, description of the pictogram can be obtained easily.

According to an aspect, the terminal device may further include a display device, and a display control unit configured to cause the display device to display a guide indicating the range in which recognition is performed by the recognition unit, in an operating mode in which the image capture device performs image capture. According to this configuration, in capturing an image, a user can select a display element of which the user desires to learn meaning. Thus, convenience is improved for a user.

According to an aspect, the descriptive information acquisition unit may acquire the descriptive information from an external storage device via the Internet. According to this configuration, the descriptive information is acquired via the Internet immediately before presentation of the descriptive information. Accordingly, the latest descriptive information can be presented to a user.

According to an aspect, the descriptive information may further include a Uniform Resource Locator (URL) of a Web page associated with the display element, and the presentation unit may further present a hyperlink to the Web page. According to this configuration, a user can easily obtain more detailed description of a display element.

### Advantageous Effects of Invention

According to the present invention, a terminal device, an information processing method, and a non-transitory storage medium recording a program that enable a user to easily obtain description of a display element displayed in an electronic device can be provided.

### BRIEF DESCRIPTION OF DRAWINGS

FIG. 1 is a view illustrating an example of a terminal device according to an embodiment.

FIG. 2 is a view illustrating appearance of a blood pressure monitor according to an embodiment.

FIG. 3 is a block diagram illustrating an example of a hardware configuration of a terminal device according to an embodiment.

FIG. 4 is a block diagram illustrating an example of a software configuration of a terminal device according to an embodiment.

FIG. 5 is a view illustrating an example of an image capture screen displayed in a terminal device according to an embodiment.

FIG. 6 is a view illustrating an example of a description screen displayed in a terminal device according to an embodiment.

FIG. 7 is a flowchart illustrating an example of an information processing method executed by a terminal device according to an embodiment.

### DESCRIPTION OF EMBODIMENTS

Hereinafter, embodiments of the present invention will be described with reference to the drawings.

### Application Example

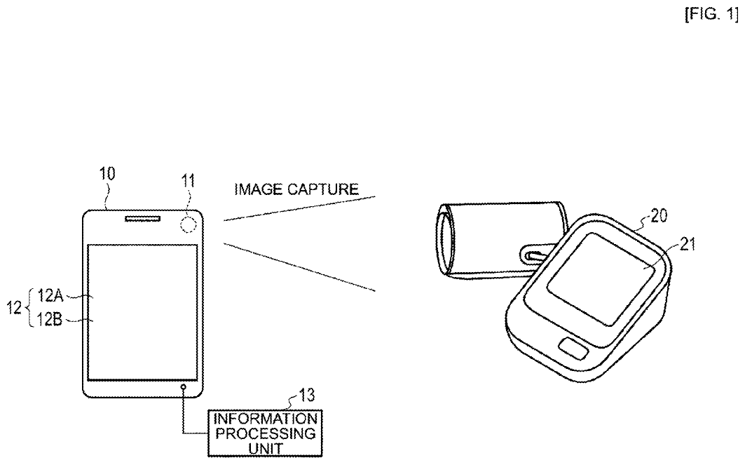

With reference to FIG. 1, an example of a scene to which the present invention is applied will be described. FIG. 1 illustrates an example of a terminal device 10 according to an embodiment. The terminal device 10 is typically a mobile device equipped with a camera 11 and a touch screen 12. The terminal device 10 may be a smart phone, a tablet personal computer (PC), a personal digital assistant (PDA), a mobile phone, or the like.

The terminal device 10 provides operation manual service related to an electronic device. The operation manual service is service of presenting, to a user, descriptive text of a display element (may also be referred to as an item) displayed on a display screen (a screen of a display device) of the electronic device. Here, the display element refers to information in display content displayed on the display screen of the electronic device, and is meaningful as an individual item. For example, a blood pressure monitor 20 that is an example of the electronic device can display the display element such as a measurement value of a systolic blood pressure, a measurement value of diastolic blood pressure, a date, time, a pictogram (may also simply be referred to as a “picto”), an error code, and an error message. The pictogram expresses information by using a pictorial symbol. The error code is a code assigned to a type (content) of error that may occur in the blood pressure monitor 20. For example, a user may not know what a pictogram displayed on a display screen 21 of the blood pressure monitor 20 means, and may wish to know the meaning of the pictogram. In that case, a user can capture an image of the blood pressure monitor 20 by using the camera 11 of the terminal device 10, and accordingly, the user can obtain descriptive text of the pictogram.

The terminal device 10 includes an information processing unit 13 in addition to the camera 11 and the touch screen 12.

The camera 11 captures an image of a target object and generates a captured image. The target object is an electronic device equipped with a display device. The electronic device is a personal or household health device, but is not limited to this. Examples of the health device include a measurement device that measures an indicator related to a human body such as a blood pressure monitor, an electrocardiograph, a scale, a body composition meter, a pedometer, and a sleep monitor, and a treatment device that performs treatment on a human body such as a transcutaneous electrical nerve stimulation (TENS) massage device. Additionally, the electronic device may also be a phone, an audio device, a remote control of an air conditioner, or the like. Additionally, the electronic device may be a device used in an office, a hospital, a factory, and the like. In the example illustrated in FIG. 1, the target object is the blood pressure monitor 20 for household. The camera 11 corresponds to an “image capture device” of the present invention.

The touch screen 12 includes a display device 12A and a touch panel 12B provided on a screen (a liquid crystal panel, for example) of the display device 12A. The display device 12A receives image data from the information processing unit 13, and displays an image according to the image data. The touch panel 12B detects a position (a contact position) on the screen at which an object such as a finger comes into contact, and outputs an operation signal indicating the contact position to the information processing unit 13. The information processing unit 13 determines content of an operation performed by a user, based on the image data provided to the display device 12A and the operation signal received from the touch panel 12B.

The information processing unit 13 receives the captured image from the camera 11. Based on the received captured image, the information processing unit 13 recognizes a display element displayed on the display screen 21 of the blood pressure monitor 20, and causes the display device 12A to display descriptive information related to the recognized display element.

As described above, the terminal device 10 according to the present embodiment captures an image of the blood pressure monitor 20 by using the camera 11, recognizes a display element displayed in the blood pressure monitor 20, and presents, to a user, descriptive information related to the recognized display element. Thus, a user performs a simple operation including the image capture of the blood pressure monitor 20 to obtain description of a display element. Thus, a user can easily obtain description of a display element such as a pictogram displayed in the blood pressure monitor 20.

Next, a terminal device according to an embodiment will be described in detail.

### Configuration Example

### Blood Pressure Monitor

First, a blood pressure monitor that is referred to for describing a terminal device according to the present embodiment will be described.

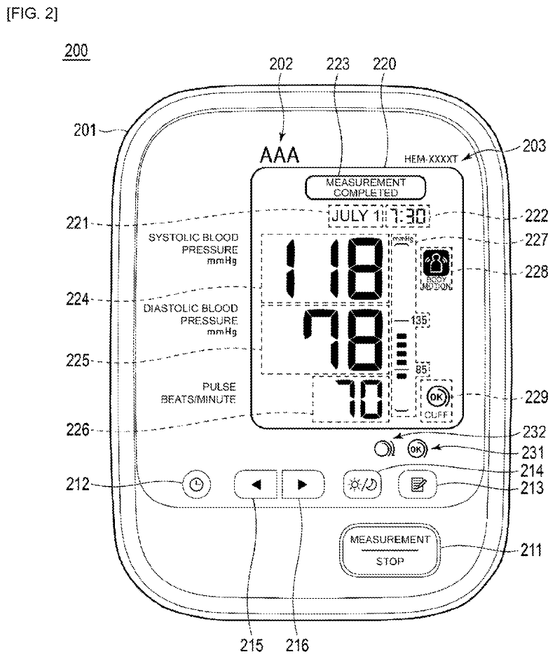

FIG. 2 illustrates appearance of a blood pressure monitor 200 as an electronic device. The blood pressure monitor 200 illustrated in FIG. 2 is an oscillometric blood pressure monitor, and is a stationary blood pressure monitor in which a body 201 and a cuff (an arm band) that is wound around an upper arm of a user are separately provided. Note that, in FIG. 2, for simplicity, the cuff and an air tube that connects the body 201 and the cuff are omitted. Additionally, since the blood pressure monitor 200 may be a generic blood pressure monitor, detailed description of the blood pressure monitor 200 will be omitted.

Brand information 202 indicating a brand (a manufacturer) of the blood pressure monitor 200 and model information 203 indicating a model of the blood pressure monitor 200 are printed in the body 201.

The body 201 includes a plurality of buttons 211 to 216 of a push type as input devices. The button 211 is a button for starting blood pressure measurement. When the button 211 is depressed, the blood pressure monitor 200 is turned on, and the blood pressure monitor 200 starts blood pressure measurement. When the button 211 is depressed during the blood pressure measurement, the blood pressure monitor 200 stops (pauses) the blood pressure measurement. The button 212 is a button for setting a clock. The button 213 is a button for viewing a record (history). The buttons 215 and 216 are used for setting the clock, selecting the record to be viewed, and the like. The button 214 is a button for viewing an average value of each of systolic blood pressure, diastolic blood pressure, and a pulse rate. The average value is, for example, an average of measurement values obtained for the most recent week. When the button 214 is depressed, an average value related to measurement performed in the morning is displayed. When the button 214 is depressed once again, an average value related to measurement performed in the evening is displayed.

The body 201 further includes a display device 220 and lamps 231 and 232. The display device 220 can display a plurality of display elements such as a date, time, a systolic blood pressure value, a diastolic blood pressure value, a pulse rate, blood pressure value level display, a body motion mark, a cuff mark, measurement completion display, and an error code. The body motion mark and the cuff mark are examples of a pictogram. In the example illustrated in FIG. 2, the display device 220 is a segmented liquid crystal display device. Thus, a region (a position) in which each of the display elements is displayed is determined. In FIG. 2, the blood pressure monitor 200 immediately after completion of blood pressure measurement is illustrated, and some of the display elements are not displayed on a screen of the display device 220.

The screen of the display device 220 includes a plurality of regions including regions 221 to 229. The date is displayed in the region 221, and the time is displayed in the region 222. The measurement completion display is displayed in the region 223. The systolic blood pressure value is displayed in the region 224, the diastolic blood pressure value is displayed in the region 225, and the pulse rate is displayed in the region 226. The blood pressure value level display is displayed in the region 227. The blood pressure value level display expresses a condition of blood pressure in 17 stages. The body motion mark is displayed in the region 228. The body motion mark indicates a body motion during measurement. In a case where no body motion is detected during measurement, the body motion mark is not lit. The cuff mark is displayed in the region 229. The cuff mark indicates whether or not the cuff is wound at appropriate intensity. The cuff mark illustrated in FIG. 2 indicates that the cuff is wound at appropriate pressure.

The error code may be displayed in the region 224 and/or the region 226. A plurality of the error codes according to the types of errors that have occurred in the blood pressure monitor 200 are prepared. For example, an error code “E1” indicates that the cuff is not properly connected to the body 201. An error code “E2” indicates that measurement has failed due to a motion of an arm or a body during the measurement. An error code “Er” indicates that the body 201 malfunctions.

When a way of winding the cuff is appropriate, the lamp 231 is lit. When the way of winding the cuff is not appropriate, the lamp 232 is lit.

The blood pressure monitor 200 is not limited to the configuration illustrated in FIG. 2. For example, the display device 220 may be a dot matrix-type display device. Additionally, although it is assumed that the blood pressure monitor 200 does not have a communication function, the blood pressure monitor 200 may include a wireless module such as a Bluetooth (registered trademark) module. The blood pressure monitor 200 may be a wearable blood pressure monitor.

### Terminal Device

### Hardware Configuration

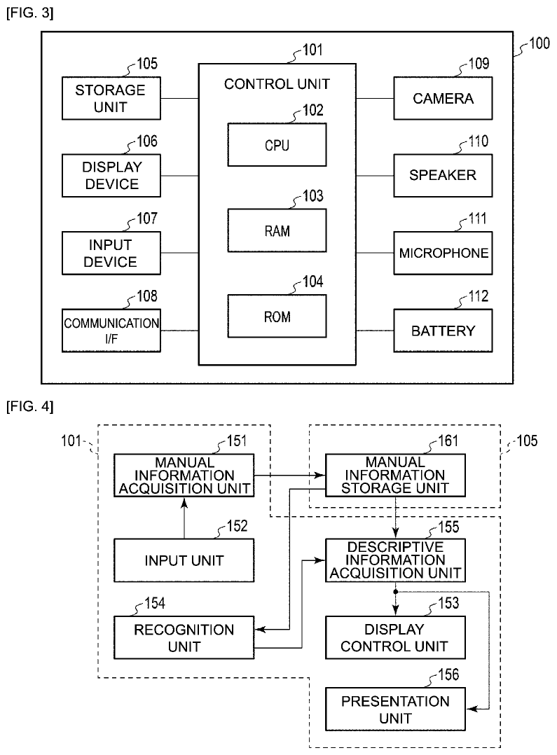

With reference to FIG. 3, an example of a hardware configuration of a terminal device 100 according to an embodiment will be described. In the example illustrated in FIG. 3, the terminal device 100 includes a control unit 101, a storage unit 105, a display device 106, an input device 107, a communication interface 108, a camera 109, a speaker 110, a microphone 111, and a battery 112.

The control unit 101 includes a central processing unit (CPU) 102, a random access memory (RAM) 103, a read only memory (ROM) 104, and the like, and controls each component. The storage unit 105 is, for example, an auxiliary storage device such as a hard disk drive (HDD) and a semiconductor memory (a flash memory, for example), and stores, in a non-transitory manner, a program to be executed by the control unit 101, settings data necessary for executing the program, and the like. A storage medium provided in the storage unit 105 is a medium that accumulates information such as a recorded program by an electrical, magnetic, optical, mechanical, or chemical action such that a computer, other devices and machines, or the like can read the information such as the recorded program. Note that some of the programs may be stored in the ROM 104.

The display device 106 displays information. Specifically, the display device 106 receives image data from the control unit 101, and displays an image according to the received image data. The display device 106 may be a liquid crystal display device or an organic electro-luminescence (EL) display. The organic EL display may also be referred to as an organic light emitting diode (OLED) display.

The input device 107 receives an operation with respect to the terminal device 100 from a user. Typically, the input device 107 includes a touch panel provided on a screen of the display device 106. The touch panel detects a position (a contact position) on the screen of the display device 106 at which an object such as a finger comes into contact, and outputs an operation signal indicating the contact position to the control unit 101. The control unit 101 determines content of an operation performed by a user, based on the image data provided to the display device 106 and the operation signal received from the touch panel. The touch panel can be, for example, a capacitive touch panel. The input device 107 may further include a button of a push type.

The communication interface 108 is an interface for communicating with an external device. The communication interface 108 sends information to or receives information from the external device. The communication interface 108 includes, for example, a wireless module including an antenna. In an example, the communication interface 108 includes a Long Term Evolution (LTE) (registered trademark) module and a Bluetooth module. Accordingly, the terminal device 100 can use the LTE module to communicate with a device such as a Web server via a mobile communication network, or can use the Bluetooth module to directly communicate with other terminal devices owned by a user. Note that the communication interface 108 may include a terminal such as a micro universal serial bus (USB) connector.

The camera 109 captures an image of a target object to generate a captured image, and outputs data of the captured image to the control unit 101. The camera 109 includes, for example, a charge coupled device (CCD) or a complementary metal oxide semiconductor (CMOS) sensor, and an image processing circuit that generates a captured image, based on an output of the CCD or the CMOS sensor. The camera 109 corresponds to the “image capture device” of the present invention. Note that at least a portion of processing performed by the image processing circuit may be performed by the control unit 101. In this case, the “image capture device” of the present invention is realized by the camera 109 and the control unit 101.

The speaker 110 converts an acoustic signal supplied from the control unit 101 into sound. The microphone 111 converts sound into an electrical signal. The microphone 111 enables a user to perform an operation (inputting a character, for example) with respect to the terminal device 100 by using voice.

The battery 112 supplies power to each of the components. The battery 112 is, for example, a rechargeable battery.

Note that, as for a specific hardware configuration of the terminal device 100, components can be omitted, replaced, or added as appropriate according to the embodiments. For example, the control unit 101 may include a plurality of processors.

### Software Configuration

With reference to FIG. 4, an example of a software configuration of the terminal device 100 will be described. In the example illustrated in FIG. 4, the terminal device 100 includes a manual information acquisition unit 151, an input unit 152, a display control unit 153, a recognition unit 154, a descriptive information acquisition unit 155, a presentation unit 156, and a manual information storage unit 161. The manual information acquisition unit 151, the input unit 152, the display control unit 153, the recognition unit 154, the descriptive information acquisition unit 155, and the presentation unit 156 execute the following processing by the control unit 101 of the terminal device 100 executing a program stored in the storage unit 105. When the control unit 101 executes the program, the control unit 101 unfolds the program in the RAM 103. Then, the control unit 101 causes the CPU 102 to interpret and execute the program unfolded in the RAM 103 to control each of the components. The manual information storage unit 161 is realized by the storage unit 105.

The manual information acquisition unit 151 acquires manual information related to the blood pressure monitor 200, and causes the manual information storage unit 161 to store the manual information. The manual information includes descriptive information related to each of the display elements that can be displayed by the blood pressure monitor 200, and image data of each of the display elements. The descriptive information related to each of the display elements includes descriptive text of each of the display elements. The descriptive information may further include a Uniform Resource Locator (URL) of a Web page associated with each of the display elements. For example, when a user inputs the model information (HEM-XXXXT, for example) of the blood pressure monitor 200 by using the input device 107, the manual information acquisition unit 151 acquires manual information associated with the model information input, from a server on the Internet via the communication interface 108.

The input unit 152 receives a user input. The user input is, for example, an instruction to execute image capture in an image capture mode in which the camera 109 performs image capture, the model information, and the like. The model information is provided to the manual information acquisition unit 151. For example, the input unit 152 receives an operation signal from the touch panel, receives, from the display control unit 153, display content information indicating content of an image displayed by the display device 106, and identifies content of an instruction input by a user, based on the operation signal and the display content information.

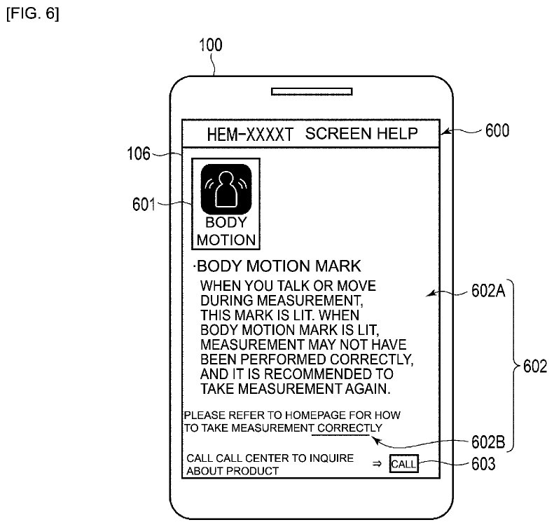

The display control unit 153 controls the display device 106. The display control unit 153 causes the display device 106 to display an image. For example, the display control unit 153 causes the display device 106 to display a description screen including the descriptive information (a description screen 600 illustrated in FIG. 6, for example). In the image capture mode, the display control unit 153 may cause the display device 106 to display a guide indicating the range in which a display element of which a user desires to learn meaning is to fit. This range corresponds to the recognition range in which recognition by the recognition unit 154 described below is performed.

The recognition unit 154 receives a captured image from the camera 109. The recognition unit 154 recognizes, based on the captured image, a display element displayed on the display screen of the blood pressure monitor 200. Specifically, the recognition unit 154 extracts, from the captured image, a partial image corresponding to the recognition range, and recognizes the display element displayed by the display device 220 of the blood pressure monitor 200, based on comparison between the partial image and the image data in the manual information stored in the manual information storage unit 161. The display element to be recognized in the present embodiment is, for example, a pictogram, an error code, and/or an error message. For example, the recognition unit 154 determines whether or not the extracted partial image includes the display element, by performing matching between the extracted partial image and the image data of each of the display elements. The recognition unit 154 outputs a recognition result including information indicating the recognized display element. Note that the recognition unit 154 may perform image recognition using artificial intelligence (AI) technology such as a neural network.

The descriptive information acquisition unit 155 acquires descriptive information related to the display element recognized by the recognition unit 154. Specifically, the descriptive information acquisition unit 155 extracts the descriptive information related to the display element recognized by the recognition unit 154, from the manual information stored in the manual information storage unit 161.

The presentation unit 156 presents, to a user, the descriptive information acquired by the descriptive information acquisition unit 155. In the present embodiment, the presentation unit 156 is the display control unit 153, and causes the display device 106 to display descriptive text of the display element. The presentation unit 156 may cause the display device 106 to display a hyperlink to a Web page associated with the display element. In another embodiment, the presentation unit 156 may synthesize the descriptive text into voice and may output the descriptive text through the speaker 110. Note that the descriptive text may be displayed by the display device 106 and also output as voice through the speaker 110.

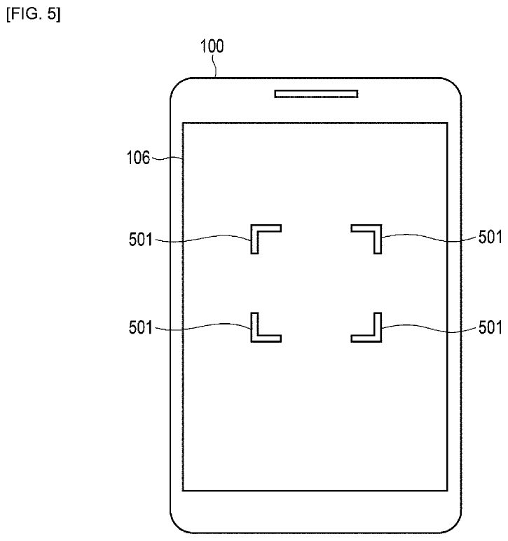

FIG. 5 illustrates an example of a guide displayed by the display device 106 in the image capture mode. As illustrated in FIG. 5, the display control unit 153 causes the display device 106 to display a frame 501 as the guide, and an image to be captured by the camera 109. In FIG. 5, the image to be captured by the camera 109 is not illustrated. A user captures an image of the blood pressure monitor 200 such that a specific display element (the body motion mark, for example) fits within the frame 501. Accordingly, descriptive information related to the specific display element is presented.

Note that the display control unit 153 may display a pointer for selecting a display element of which a user desires to learn meaning. Accordingly, even in a case where two display elements are positioned close to each other, it becomes easy to select one of the display elements. Additionally, the display control unit 153 may not display the guide in the image capture mode. In this case, a user selectively captures an image of a specific display element by using, for example, a zoom function of the camera 109.

The description screen 600 includes an image 601 of the display element recognized by the recognition unit 154, and descriptive information 602 related to the display element. In the example illustrated in FIG. 6, the descriptive information 602 includes descriptive text 602A related to the display element and a hyperlink 602B to a Web page associated with the display element. In response to a user tapping the hyperlink 602B, a browser starts and a Web page is displayed in the browser. A moving image may be embedded in a Web page. For example, a Web page associated with the cuff mark includes a moving image describing a method for attaching the cuff. Accordingly, a user can obtain more detailed description than description related to each of the display elements that is generally provided in an operation manual (an electronic manual, for example).

The description screen 600 may further include a button 603 for calling an operator. Typically, the button 603 is initially inactive, and after a user taps the hyperlink 602B, the button 603 is activated. In response to the user tapping the button 603 in an active state, the control unit 101 of the terminal device 100 makes a call to a call center related to the blood pressure monitor 200. To prevent a user from calling an operator several times in succession, the button 603 may be inactive or may not be displayed until predetermined time elapses after the button 603 has been depressed once.

Note that, in the present embodiment, the example in which any of the functions of the terminal device 100 is realized by a general-purpose processor is described. However, some or all of the functions may be realized by a single or a plurality of dedicated processors.

### Operation Example

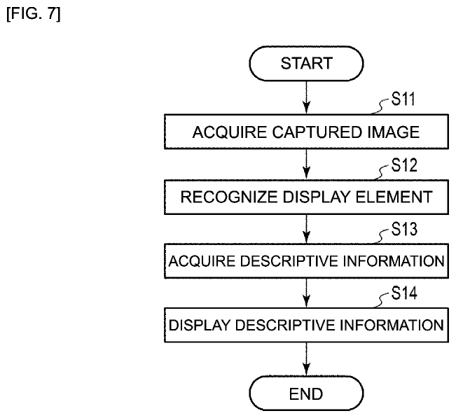

FIG. 7 illustrates an operation flow performed when the terminal device 100 provides the operation manual service.

At step S11 in FIG. 7, the control unit 101 of the terminal device 100 acquires a captured image from the camera 109. For example, a user starts operation manual application on the terminal device 100 to find out meaning of the body motion mark displayed on the display screen of the blood pressure monitor 200. When the operation manual application starts, the terminal device 100 becomes in the image capture mode. A user captures, by using the camera 109, an image of the blood pressure monitor 200 being in a state where the body motion mark is displayed on the display screen, and the camera 109 outputs the captured image to the control unit 101. Specifically, the user captures the image of the blood pressure monitor 200 by using the camera 109 such that the body motion mark fits within the frame 501 corresponding to the recognition range.

At step S12, the control unit 101 operates as the recognition unit 154, and recognizes, based on the acquired captured image, the display element displayed on the display screen of the blood pressure monitor 200. For example, the control unit 101 extracts, from the captured image, a partial image corresponding to the recognition range, performs matching between the partial image and a plurality of pieces of the image data, and identifies the display element corresponding to image data having the highest degree of similarity. For example, the control unit 101 recognizes that the body motion mark is displayed on the display screen of the blood pressure monitor 200.

At step S13, the control unit 101 operates as the descriptive information acquisition unit 155, and acquires descriptive information related to the recognized display element. For example, the control unit 101 extracts descriptive information associated with the body motion mark from the manual information stored in the storage unit 105.

At step S14, the control unit 101 operates as the presentation unit 156, and causes the display device 106 to display descriptive text in the acquired descriptive information. For example, the control unit 101 causes the display device 106 to display a description screen including descriptive text of the body motion mark (the description screen 600 illustrated in FIG. 6, for example). The description screen may further include a hyperlink to a Web page associated with the body motion mark.

In this way, the terminal device 100 displays the descriptive information related to the display element displayed in the blood pressure monitor 200.

Note that the processing procedure illustrated in FIG. 7 is merely an example, and the processing procedure or contents of each kind of the processing can be modified as appropriate. For example, when the control unit 101 recognizes a plurality of the display elements at step S12, the control unit 101 may cause the display device 106 to display descriptive information related to each of the display elements. Alternatively, the control unit 101 may cause the display device 106 to display a list of the plurality of recognized display elements to prompt a user to select one of the display elements. In this case, the display device 106 displays descriptive information related to the display element selected by the user.

### Effects

The terminal device 100 described above recognizes a display element in display content, based on a captured image generated by the camera 109 capturing an image of the blood pressure monitor 200 being in a state where the display content is displayed on a display screen, acquires descriptive information including descriptive text of the recognized display element, and displays the descriptive information by the display device 106. Accordingly, a user can obtain descriptive information related to a display element by performing a simple operation including the image capture of the blood pressure monitor 200. In other words, a user can reach descriptive information related to a display element by a small number of procedures. Thus, a user can quickly and easily learn meaning of a display element displayed in an electronic device.

A user captures an image by using the camera 109 such that a display element of which the user desires to learn meaning fits within the guide (the frame 501 illustrated in FIG. 5, for example). That is, the selection of a display element of which a user desires to learn meaning is performed by the image capture using the camera 109. Thus, even as for a display element such as a pictogram that is difficult to express by using a keyword, description of the display element can be obtained easily.

### Modified Examples

Note that the present invention is not limited to the embodiments described above.

For example, the manual information acquisition unit 151 and the manual information storage unit 161 are not necessarily required. In an embodiment in which the manual information acquisition unit 151 and the manual information storage unit 161 are not provided, each time a terminal device 100 provides operation manual service, the terminal device 100 accesses, via the Internet, a server that holds manual information. For example, a recognition unit 154 acquires, from the server via a communication interface 108, image data associated with model information, and performs recognition, based on the acquired image data and a captured image. A descriptive information acquisition unit 155 acquires descriptive information related to a display element recognized by the recognition unit 154. For example, the descriptive information acquisition unit 155 sends, to the server via the communication interface 108, a request signal of requesting the descriptive information related to the display element recognized by the recognition unit 154, and receives, from the server via the communication interface 108, the descriptive information related to the display element. The descriptive information may be updated at any timing. Each time the operation manual service is provided, the descriptive information is acquired, and accordingly, the latest descriptive information can be presented to a user. Further, since it is not necessary for the terminal device 100 to hold the manual information, a storage resource can be saved.

Additionally, in a case where the terminal device 100 is configured to acquire the descriptive information via the Internet each time the operation manual service is provided, a manufacturer can understand what kind of information is requested by a user, based on a request signal collected by the server. In other words, a manufacturer can obtain information useful for improving a product (the blood pressure monitor 200 in this example). The request signal may include information related to a user, together with information identifying a display element selected by the user. The information related to a user includes, for example, gender and/or an age of the user.

In the embodiment described above, a user inputs the model information of the blood pressure monitor 200. In an embodiment, the terminal device 100 may include a model identification unit that identifies a model of the blood pressure monitor 200 from a captured image. The model identification unit executes predetermined processing by the control unit 101 executing a program stored in the storage unit 105.

In an example, as illustrated in FIG. 2, the model information 203 is printed in the vicinity of the display screen of the blood pressure monitor 200, and in a case where an image of the blood pressure monitor 200 is captured such that any of the display elements fits within the guide (the frame 501 illustrated in FIG. 5, for example), the model information 203 is in the captured image. The model identification unit performs character recognition on the captured image, and accordingly identifies a model of the blood pressure monitor 200. In place of or in addition to the model information 203, an identification code for identifying a model may be provided in the vicinity of the display screen of the blood pressure monitor 200. The identification code may be, for example, a two-dimensional code. The model identification unit decodes the identification code in a captured image, and accordingly identifies a model of the blood pressure monitor 200. The identification code may include a command for starting operation manual application. In this case, when the identification code is read by the camera 109, the control unit 101 starts the operation manual application, and also identifies a model of the blood pressure monitor 200.

In the manual information storage unit 161, the manual information is stored in association with each of a plurality of models. The recognition unit 154 reads out, from the manual information storage unit 161, manual information associated with a model identified by the model identification unit described above, extracts, from a captured image, a partial image corresponding to the recognition range, and recognizes a display element displayed by a display device of an electronic device, based on comparison between the extracted partial image and image data in the read-out manual information. The descriptive information acquisition unit 155 acquires, from the manual information storage unit 161, descriptive information related to the display element recognized by the recognition unit 154. The presentation unit 156 presents, to a user, the descriptive information acquired by the descriptive information acquisition unit 155.

In a case where the terminal device 100 includes the model identification unit, it is not necessary for a user to input the model information. Thus, convenience is improved for a user.

The operation manual service may be a function of health management application for recording and managing blood pressure measurement results and the like. For example, a user interface of the health management application is provided with a help button for executing the operation manual service. When a user taps the button, the processing described with reference to FIG. 7 is executed.

In short, the present invention is not limited to the embodiments described above as they are, and the components can be modified and embodied without departing from the gist of the present invention in a stage of implementation. Additionally, various inventions can be formed by appropriately combining a plurality of the components disclosed in the embodiment described above. For example, some of the components may be omitted from all the components described in the embodiments. Further, the components of the different embodiments may be combined appropriately.

### Supplementary Notes

A portion or all of the embodiments can also be described as described in the following supplementary notes in addition to the claims, but are not limited to this.

A terminal device (100) includes

an image capture device (109) configured to capture an image of an electronic device to generate a captured image, the electronic device being in a state where display content including a display element is displayed on a display screen,
a recognition unit (154) configured to recognize, from the captured image, the display element in the display content,
a descriptive information acquisition unit (155) configured to acquire descriptive information including descriptive text of the display element, and
a presentation unit (156) configured to present the descriptive text in the descriptive information.

### REFERENCE SIGNS LIST

10 Terminal device

11 Camera

12 Touch screen

12A Display device

12B Touch panel

13 Information processing unit

20 Blood pressure monitor

21 Display screen

100 Terminal device

101 Control unit

102 CPU

103 RAM

104 ROM

105 Storage unit

106 Display device

107 Input device

108 Communication interface

109 Camera

110 Speaker

111 Microphone

112 Battery

151 Manual information acquisition unit

152 Input unit

153 Display control unit

154 Recognition unit

155 Descriptive information acquisition unit

156 Presentation unit

161 Manual information storage unit

200 Blood pressure monitor

201 Body

202 Brand information

203 Model information

211 to 216 Button

220 Display device

1. A terminal device comprising:
an image capture device configured to capture an image of an electronic device to generate a captured image, the electronic device being in a state where display content including a display element is displayed on a display screen; and
one or more processors configured to:
recognize, from the captured image, the display element in the display content;
acquire model information indicating a model of the electronic device;
acquire descriptive information including descriptive text of the display element and associated with the model information; and
present the descriptive text in the descriptive information.

2. The terminal device according to claim 1, wherein
the display element is a pictogram.

3. The terminal device according to claim 1 further comprising:
a display device;
wherein the one or more processors are further configured to cause the display device to display a guide indicating the range in which recognition is performed, in an operating mode in which the image capture device performs image capture.

4. The terminal device according to claim 1, wherein
the one or more processors are further configured to acquire the descriptive information from an external storage device via the Internet.

5. The terminal device according to claim 1, wherein
the descriptive information further includes a Uniform Resource Locator (URL) of a Web page associated with the display element, and
the one or more processors are further configured to present a hyperlink to the Web page.

6. An information processing method executed by a terminal device including an image capture device configured to capture an image of an electronic device to generate a captured image, the electronic device being in a state where display content including a display element is displayed on a display screen, the information processing method comprising processes of:
recognizing, from the captured image, the display element in the display content;
acquiring model information indicating a model of the electronic device;
acquiring descriptive information including descriptive text of the display element and associated with the model information; and
presenting the descriptive text in the descriptive information.

7. A non-transitory storage medium recording a program for causing the one or more processors to perform functions provided in the terminal device according to claim 1.
